(19) United States
(12) Patent Application Publication
(10) Pub. No.: US 2026/0354789 A1
(43) Pub. Date: Nov. 12, 2026

(54) BROAD-FIELD DOPPLER ULTRASOUND OF CEREBRAL BLOOD FLOW TO IMPROVE THE ADMINISTRATION OF TREATMENTS

(71) Applicant: NUTECH VENTURES, Lincoln, NE (US)

(72) Inventors: Greg Bashford, Lincoln, NE (US); Benjamin Hage, Lincoln, NE (US)

(21) Appl. No.: 19/127,974

(22) PCT Filed: Nov. 10, 2023
(86) PCT No.: PCT/US2023/079323

§ 371 Date: May 7, 2025

Related U.S. Application Data
(60) Provisional application No. 63/383,410, filed on Nov. 11, 2022.

(51) Int. Cl. A61B 8/08; A61B 8/00; A61B 8/06

(52) U.S. Cl. CPC A61B 8/0808; A61B 8/06; A61B 8/463; A61B 8/488

(57) ABSTRACT

Devices and methods are provided for rapid determination and feedback of cerebral blood flow (CBF) while performing an emergency procedure. The device may be portable and include a Doppler ultrasound transducer, a handle attached to the transducer, and a display integral with the handle. The transducer may use a broad-field broad acoustic beam for faster acquisition and processing of the desired CBF data. The display may present information indicative of the condition of CBF in a subject determined from the Doppler ultrasound signals. The device may also be configured to provide audio and/or visual feedback to improve the quality of an emergency procedure, such as cardiopulmonary resuscitation (CPR).

### TECHNICAL FIELD

The present disclosure relates to devices and methods for analyzing blood flow in blood vessels and, more particularly, devices and methods for analyzing cerebral blood flow to improve the quality of medical treatments.

### BACKGROUND

Survival rates of out-of-hospital cardiac arrest are dismal. Despite advances in cardiopulmonary resuscitation (CPR) in the last 20 years, rates of survival to discharge (typically 5% to 9%) are discouraging, and only 3% to 7% recover their pre-cardiac arrest neurological health. CPR is the primary emergency technique employed during cardiac arrest. The quality of CPR is an important and time-critical factor affecting patient outcomes. “High-quality” CPR may include a compression rate between 80 and 120 beats per minute and a compression depth of 40 millimeters (mm) and 60 mm. However, the optimal values of these variables may vary significantly between individuals and demographics. Therefore, CPR needs to be “individualized” or targeted based on hemodynamic targets. For example, in animal studies in which CPR was performed targeting a coronary perfusion pressure of greater than 20 millimeters of mercury (mmHg) and a systolic blood pressure greater than 100 mmHg rather than targeting a 50 mm compression depth, there was significantly improved survival at 45 minutes and at 24 hours, which strongly indicates an advantage of targeting CPR to the individual.

The main problem preventing targeting and personalization of CPR is monitoring blood flow where it matters—in the cerebral tissue, where it is called cerebral blood flow (CBF). The goal of CPR is to “achieve optimal cardiac output with maximal coronary and cerebral perfusion.” However, those that perform CPR in pre-hospital settings (e.g., paramedics and emergency medical technicians) currently have no means of directly measuring CBF, leaving open the possibility that, although providers may be providing compressions at the rate and compression depth recommended by the American Heart Association (AHA), CBF remains low or nonexistent. Additionally, a target rate and compression depth for CPR may vary based on physiological factors and demographics.

Currently, no portable, user-friendly device exists that can measure CBF during CPR in out-of-hospital settings, and, as a result, CPR decision may be made from pulse palpation, continuous-wave Doppler ultrasound of the femoral artery, capnography, and monitoring other vital signs. However, by their nature, these methods may be slow (e.g., tens of minutes) to respond to alterations in CPR, and thus may not be suitable for emergency use. Additionally, these methods may often be performed on peripheral arteries (i.e., not arteries feeding the brain) not directly related to cerebral perfusion, and thus first responders may not be able to readily determine whether the brain itself is being perfused.

### SUMMARY

A portable device utilizing a large, broad-field acoustic beam enables more rapid, real-time treatment decisions to be based directly on CBF measurements. The device may have many advantages in time-sensitive applications, such as CPR. For example, alterations in diastolic blood flow velocity could inform decisions, such as whether to increase CPR compression rate/depth, when to administer more intravenous adrenaline, and when to add normal saline to increase volume loading. The device may also provide indications for other applications such as, for example, trauma and shock management, brain injury victims, and detecting the conditions of ischemic stroke. The informing of these and other response decisions may lead directly to improvement in patient management and improved survival and neurological outcomes.

Accordingly, Applicant has recognized a need for assemblies, devices, systems, and methods for directly measuring CBF conditions, which may rapidly inform decisions during emergency CPR, as well as other treatments. The present disclosure provides devices and methods that may address one or more of the above-referenced drawbacks, as well as other possible drawbacks.

As referenced above, it may be desirable to provide enhanced assemblies, devices, systems, and methods for measuring CBF in emergency settings for CPR and/or other treatments. For example, the condition of CBF in a patient may be determined using a device with a broad-field Doppler ultrasound transducer to rapidly determine information, such as anterograde and retrograde blood flow velocities, and calculate flow metrics and other parameters to inform decision making and improve patient outcomes.

In some embodiments, a device for monitoring CBF may include a transducer configured to generate ultrasound signals indicative of CBF conditions in a subject, a handle attached to the transducer configured for an operator to manipulate the transducer with one hand, and a display integral with the handle configured to present one or more quality parameters determined from the ultrasound signals. The one or more quality parameters determined from the ultrasound signals may be indicative of the presence and strength of systolic, diastolic, and/or average flow in the subject. The device may also include a speaker configured to generate an audio output for the operator, and the audio output may be modulated by the Doppler spectrum envelope to provide an auditory representation of the Doppler spectrum. The device may be portable and configured to provide the CBF conditions both in and outside of hospital locations (e.g., medical treatment facilities).

In some embodiments, the device may be configured to calculate and display the one or more quality parameters determined from a specified ultrasound signal of the ultrasound signals from the transducer within 3 seconds or less of the generation of the specified ultrasound signal. The quality measurements may be used by the operator to alter a set of CPR parameters being administered to the subject, the CPR parameters including at least one of: a compression rate, a compression depth, duty cycle, inter-compression pause time, and release velocity.

In some embodiments, the device may include a processing unit configured to process and perform calculations on the ultrasound signals from the transducer, as well as an energy storage device as a power source for the device. The device may also include a communications module configured to communicate the ultrasound signals to a receiver remote from the device.

In some embodiments, a method for performing CPR based on monitored cerebral blood flow (CBF) may include performing CPR on a subject based on a first set of CPR parameters and positioning an ultrasound device with an ultrasound transducer at a first position on the patient. Measuring of a Doppler spectrum may be performed with the ultrasound transducer to generate a first set of ultrasound signals. The method may include calculating flow metrics and diastolic sufficiency indicative of a first condition of CBF and displaying a first set of quality parameters on a display of the ultrasound device based on the first condition of CBF. Displaying of the one or more quality parameters may involve displaying at least one of anterograde blood flow velocities, retrograde blood flow velocities, and plots of transcranial Doppler spectral data. The method may further include determining a second set of CPR parameters based at least in part on the first set of quality parameters and performing CPR on the subject based on the second set of CPR parameters.

The ultrasound device may be portable and configured to provide the CBF conditions in hospital settings or out-of-hospital locations. The ultrasound device may have a handle with an integrated display, and a speaker configured to generate an audio output that provides an auditory representation of the Doppler spectrum. The ultrasound device may include, for example, a processor to perform the calculating of the flow metrics and diastolic sufficiency. The ultrasound device may further include a power source for providing power to one or more of the processing unit, the display, and the ultrasound transducer.

In some examples, the method may include communicating the ultrasound signals to a receiver remote from the ultrasound device, receiving the communicated ultrasound signals, and displaying a display output, such as a data structure, on an external display in communication with the receiver. The data structure may include at least one of information relating to condition of CBF from the communicated ultrasound signals and a lookup table comprising a set of known hemodynamic targets and CPR parameters.

In some embodiments, the method may include generating a second set of ultrasound signals responsive to the CPR performed at the second set of CPR parameters and calculating flow metrics and diastolic sufficiency indicative of a second condition of CBF. The method may also involve displaying a second set of quality parameters on the display based on the second condition of CBF. Additionally, the method may include determining a third set of CPR parameters based at least in part on the change between the first set of quality parameters and the second set of quality parameters and performing CPR on the subject based on the third set of CPR parameters.

In some embodiments, the method may involve positioning the ultrasound device at a second position on the patient, different from the first position, and generating another set of ultrasound signals indicative of a second condition of CBF at the second position responsive to the CPR performed. The method may then include calculating and displaying another set of quality parameters on the display based on the second condition of CBF. Further, the method may also include determining, based at least in part on a comparison of the first condition of CBF at the first position and the second condition of CBF at the second position, a difference in diastolic flow between first position and the second position. This difference may be used to inform a CPR performer to change CPR parameters.

Still other aspects and advantages of these exemplary embodiments and other embodiments, are discussed in detail herein. Moreover, it is to be understood that both the foregoing information and the following detailed description provide merely illustrative examples of various aspects and embodiments and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a schematic perspective view an example device for monitoring CBF in a subject, according to embodiments of the disclosure.

FIG. 2 is a schematic top view of the example device of FIG. 1, according to embodiments of the disclosure.

FIG. 3 is a schematic side view of the example device of FIG. 1, according to embodiments of the disclosure.

FIG. 4 is a schematic front view of the example device of FIG. 1, according to embodiments of the disclosure.

FIG. 5 is a schematic rear view of the example device of FIG. 1, according to embodiments of the disclosure.

FIG. 6 is a view of an example device for monitoring CBF, according to embodiments of the disclosure.

FIG. 7 is a schematic view of an example device using a broad-field beam on a subject's brain, according to embodiments of the disclosure.

FIG. 8 is a view of a portion of an example display output of the device, according to embodiments of the disclosure.

FIG. 9 is a view of a portion of another example display output of the device, according to embodiments of the disclosure.

FIG. 10 is a view of a portion of a further example display output of the device, according to embodiments of the disclosure.

FIG. 11 is a flow diagram of an example method for altering CPR parameters using feedback from monitored CBF, according to embodiments of the disclosure.

FIG. 12 is another flow diagram of an example method for performing CPR using feedback from monitored CBF, according to embodiments of the disclosure.

### DETAILED DESCRIPTION

The drawings include like numerals to indicate like parts throughout the several views, the following description is provided as an enabling teaching of exemplary embodiments, and those skilled in the relevant art will recognize that many changes may be made to the embodiments described. It also will be apparent that some of the desired benefits of the embodiments described may be obtained by selecting some of the features of the embodiments without utilizing other features. Accordingly, those skilled in the art will recognize that many modifications and adaptations to the embodiments described are possible and may even be desirable in certain circumstances. Thus, the following description is provided as illustrative of the principles of the embodiments and not in limitation thereof.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, in particular, to mean “including but not limited to,” unless otherwise stated. Thus, the use of such terms is meant to encompass the items listed thereafter, and equivalents thereof, as well as additional items. The transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements. As used herein, “real-time” refers to a delay one minute or less (e.g., on the order of 60 seconds, 30 seconds, 3 seconds, or less) between collecting measurement data and providing analysis and/or recommendations based on the collected measurement data.

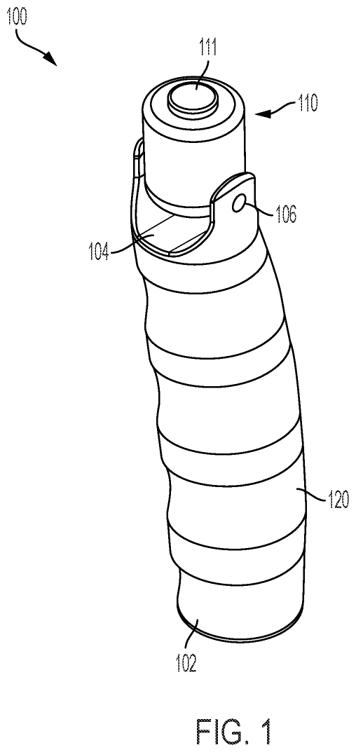

Referring to the figures, FIGS. 1-5 illustrate various schematic views of an embodiment of an example device 100 for monitoring CBF in a subject. The device 100 may include a handle 120 having a substantially cylindrical profile for grasping. The device 100 may be lightweight and small enough to be easily manipulated by an operator using one hand. In some examples, a sensor or transducer 110 can be mounted on a pivot 106 for ease of orientation relative to the handle 120. The pivot 106 may permit articulation of the transducer 110 along an axis different from (e.g., orthogonal to) an axis of the handle 120. For example, the pivot 106 may allow a sensor head 111 of the transducer 110 to be placed flush with a subject's head or other anatomical curvature while the handle 120 is maintained in an ergonomic position for the operator. The handle 120 may have a contoured grip and may be, for example, constructed from a polymeric material, such as acrylonitrile butadiene styrene (ABS) or other materials known in the art, and formed by injection molding, overmolding, and/or other suitable process.

The device 100 may include an ultrasound transducer 110 capable of transcranial Doppler (TCD) ultrasound measurements attached near the distal end 104 of the handle 120. Doppler ultrasound may be used to estimate the blood flow through certain body vessels local to the transducer by emitting high frequency sound waves and receiving reflected waves after they have rebounded off of circulating red blood cells. The Doppler ultrasound technique may be used, for example, to detect and diagnose blood clots, venous insufficiency, arterial occlusions, reduced circulation, aneurysms, and/or other conditions. A Doppler ultrasound technique may also be used to estimate the velocity of blood flows by measuring the rate of change in the frequency (pitch) of the TCD signals from the transducer.

Example transducer 110 may be the sound emitting and receiving component of the TCD ultrasound technique and may be configured to continuously generate ultrasound signals indicative of CBF in a subject during operation. The transducer 100 may have a cylindrical body having a diameter of, for example, between approximately 5 mm and approximately 50 mm, although other shapes and sizes may also be contemplated. The transducer 110 may be designed based on such parameters as beam spread, frequency range, and excitation power to generate TCD spectra. Control and adjustment of these parameters may allow for the calibration of an enhanced transducer 110 in which the point spread function (PSF), and thus the volume of the brain from which CBF is detected, is enlarged. This enhanced range of transducer 110 may be accomplished while maintaining a TCD beam containing sufficient acoustic energy to insonate red blood cells in the volume and keeping acoustic power below the U.S. Food and Drug Administration (FDA) limit of 720 milliwatts per square centimeter (mW/cm²). The design of an enhanced transducer 110 having a large PSF is essential for fast blood flow detection during time-sensitive events, such as CPR.

During traditional performance of CPR, the adequacy of perfusion may typically be estimated in the pre-hospital setting using pulse oximetry and capnography. However, it has been found that pulse oximetry does not correlate well with arterial oxygen content in humans or in animal models of CPR. Capnography (i.e., end-tidal carbon dioxide (CO₂)) may give information on cardiac output and adequacy of compression depth, but it does not directly measure cerebral perfusion. Other traditional technologies, including near-infrared spectroscopy and related optical techniques, may measure oxygenation of the cerebral cortex, but are not fast enough to allow for instantaneous adjustments in time-critical procedures such as CPR. Capnography, for example, may lag hemodynamic changes by about 30 seconds or more, making instantaneous adjustments in care impossible.

In contrast, the transducer 110 of example device 100 may be capable of continuously generating and processing ultrasound signals indicative of the CBF conditions at a rate rapid enough to enable real-time decisions to be based directly on the CBF measurements in emergency situations. The speed at which TCD readings may be attained and interpreted in disclosed embodiments of the device 100 may allow the device to be used in the life support protocols of first responders, such as emergency medical services and fire and rescue services. The use of example device 100 may allow for CPR decisions to be informed by rapid interpretation of CBF readings in the brain, the organ which CPR is intended to preserve. Based on CBF measurements, for example, paramedics and emergency medical service providers may adjust their technique and alter CPR parameters such as compression depth, compression rate, duty cycle, inter-compression pause time, and release velocity to maximize CBF, and thus the chance of survival. The emergency medical service providers often utilize standard equipment totes (e.g., containing a cardiac monitor/defibrillator for measuring and stimulating the heart's electrical activity, a capnograph for monitoring expired CO₂, and other common equipment) and the device 100 may therefore have a small enough footprint to be easily carried the same standard totes.

It should be noted that, although the technique discussed herein in the context of informing CPR decisions for emergency first responders, the use of the present technique for other indications and applications is also contemplated. For example, parameters obtained from real-time monitoring of CBF may provide feedback for trauma and shock management to allow for adjustments, such as fluid resuscitation. In another example, the devices and methods disclosed may also be used in monitoring traumatic brain injury victims for vasospasm and hypoperfusion to optimize treatment. In another example, the ability to rapidly measure cerebral perfusion in challenging environments may lead to point-of-care ultrasound treatments in emergency rooms for evaluating prognosis and diagnosis. In a further example, the devices and methods disclosed may also be used to detect large vessel occlusion ischemic stroke while in transport (e.g., ambulance, airlift, etc.), indicating a need to transport the patient to an endovascular thrombectomy-capable center, which may lead to improved subject survival and outcomes.

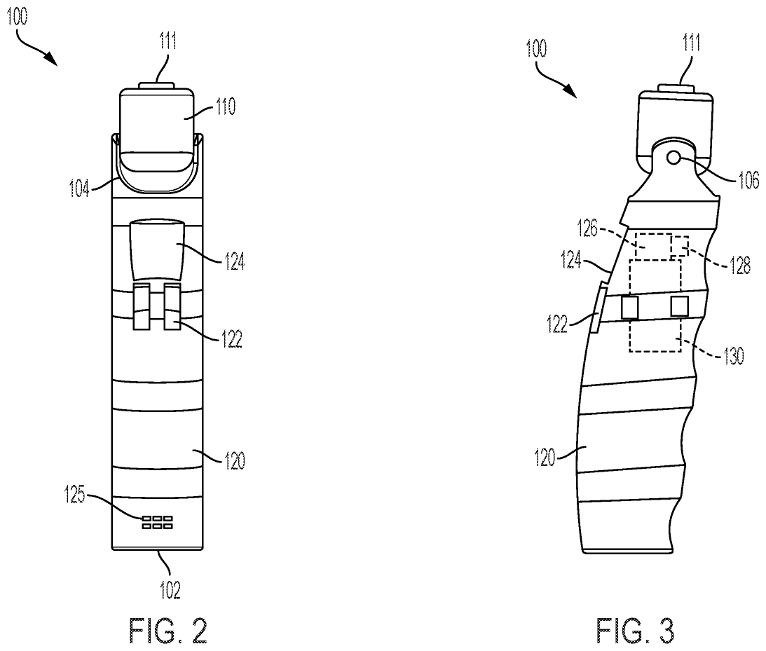

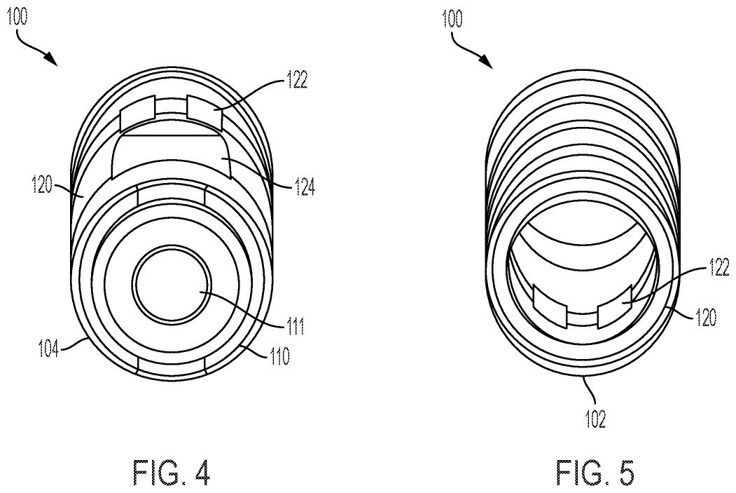

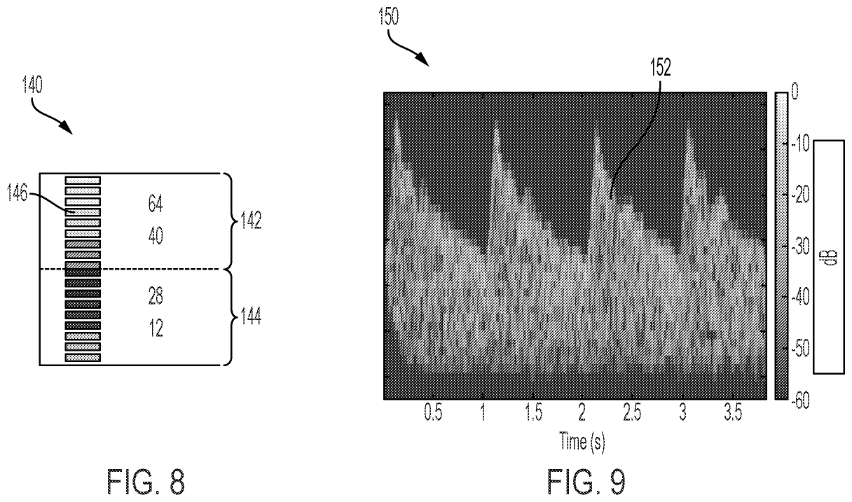

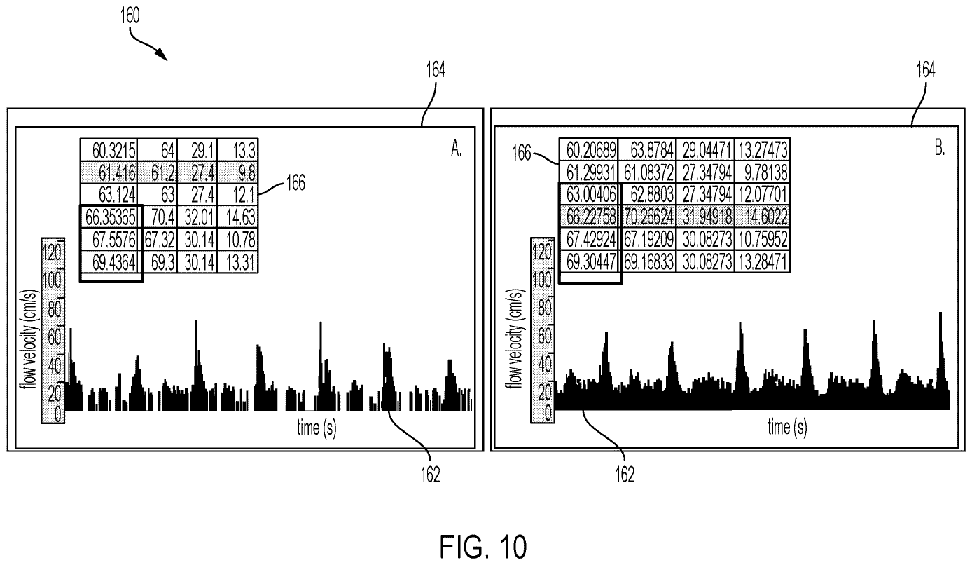

In some embodiments, an example device 100 may have a simple user interface designed for the rapid assessment of vital data. For instance, the device may have a pulse indicator, user controls, an audio output, and/or a display to control and highlight key CBF parameters. In some examples, whereas currently available handheld ultrasound transducers may lack rapid readout capabilities on the device itself, the device 100 may have an integrated display 124 on the handle 120 to provide emergency responders with immediate visual feedback (as shown in more detail in FIGS. 8 and 9). In some examples, the device 100 may have one or more control features 122 on the handle 120 for controlling and adjusting operating parameters of the device 100, as seen in FIG. 2, FIG. 3, and FIG. 4. The control features 122 may be one or more of a combination of, for example, scroll wheels, joysticks, buttons, knobs, toggles, and/or other similar implements for accepting operator inputs.

In some embodiments, the device 100 may have a processor 126 configured to process and perform calculations on the Doppler ultrasound signals. The processor 126 may also accept inputs from the control features 122 of the device 100. For example, the processor 126 may utilize the ultrasound signals to calculate flow metrics and diastolic sufficiency indicative of a current state or condition of CBF. The processor 126 may be capable of, for example, performing Fast Fourier Transformations (FFT) in the frequency domain and other algorithms on the sampled ultrasound data. In some examples, the processor may further calculate one or more quality parameters based on the condition of CBF. The quality parameters determined from the ultrasound signals may be indicative of the presence and strength of diastolic flow in a subject. In some examples, the quality parameters may be used as digital readout values on the display 124 and/or as triggers for other alerts/alarms to facilitate a determination of treatment response for an operator or operators of the device 100. The quality parameters may be used to alter a set of CPR parameters used on a subject, such as at least one of a compression rate, a compression depth, a duty cycle, an inter-compression pause time, and a release velocity.

In some embodiments, the device 100 may include an audio device 125 (FIG. 2) configured to generate an audio output for operators of the device. The audio device 125 may be, for example, a speaker or similar device that can be triggered and controlled by the processor 126. The audio device may give an audio output that provides an auditory representation of the Doppler spectrum to allow trained observers to understand the pattern of blood flow. In some examples, the audio device 125 may provide the “swish swish” output of familiar ultrasound modulations in the Doppler spectrum envelope. In some examples, the audio device 125 may generate audible beeps or spoken messages to serve as warnings, alerts, or notifications to a CPR performer.

The audio device 125 may also give an audio output based on, for example, a comparison of the one or more quality parameters with a set of known hemodynamic targets. In one example, when CBF and the determined quality parameters indicate inadequate CBF, the audio device 125 may be triggered by the processor 126 to audibly alert an operator to “Pump Faster” when compressions are determined to be too slow. Similarly, the audio device 125 may be triggered by the processor 126 to audibly alert an operator to “Pump Slower” when compressions are determined to be too fast. In a further example, if CBF remains inadequate but the compression rate is determined to be acceptable, the audio device 125 may be triggered by the processor 126 to audibly alert an operator to “Pump Deeper.”

In some examples, as shown in FIG. 3, the processor 126 may also be in communication with a communications module 128 capable of providing data in/out operations to devices and/or locations remote from the device 100. In some embodiments, the communications module may be a transmitter or antennae in communication with a remote receiver 160 (see, e.g., FIG. 10 and detailed discussion below) via a hard-wired communications network and/or a wireless communications network, which may adhere to known hard-wired and/or wireless communication protocols. The communications module 128 may be capable of communications via, for example, wireless via radio-frequency (RF) methods (e.g., Bluetooth, mobile broadband, etc.), optical methods, infrared methods, or other suitable means. The communications module 128 may allow the device to communicate with other assets within and outside of hospital settings.

The device 100 may also include an energy storage device 130 as an electrical power source. The energy storage device 130 may be used, for example, to provide electrical power to one or more of the processor 126, the display 124, the ultrasound transducer 110, the communications module 128, and the audio device 125. In some embodiments, the energy storage device 130 may be any of a number of commercially available direct current (DC) batteries. The energy storage device 130 may be disposable or rechargeable.

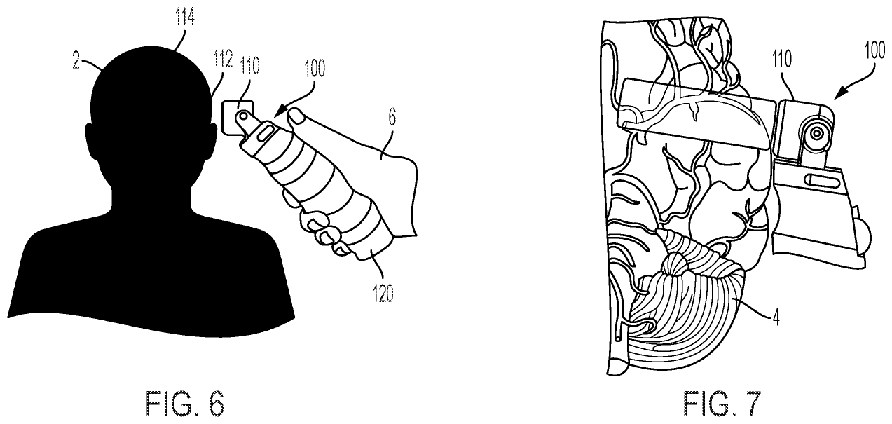

FIG. 6 shows an example placement of an embodiment of the device 100 at a location 112 proximate the skull of a subject 2. A single hand 6 of the user may hold the handle 120 clear of other obstructions while the transducer 110 is placed flush with the subject's head (e.g., flush with the external soft tissue of the subject that covers the subject's skull). Although the example positioning shown in FIG. 6 may be similar to that of other handheld ultrasound transducers that exist on the market, other designs are often intended to be portable versions of conventional ultrasound probes (i.e., narrow beam field and tight focusing), whereas the embodiments disclosed herein may have a larger, broader beam adapted for emergency use.

In some embodiments, after CBF readings are obtained at the first location 112 on the subject and determinations are made regarding a perfusion diagnosis based on the calculated flow metrics, the device may be quickly maneuvered to an alternate or second location 114 on the head of the subject. A comparison between further CBF readings taken at the second location with the CBF readings obtained at the first location may allow for a more targeted scan of certain regions of brain tissue or particular vessels of interest. The flexibility to rapidly deploy and compare CBF data at different locations may allow for useful observations in circumstances where, for example, there is an indication of a significant vessel occlusion or the subject is suffering from localized trauma. In other examples, the transducer 110 may be able to automatically, electronically “steer” or direct the beam to a different second location 114 of the subject's brain (e.g., utilizing the processor to change the angle, width, and/or other beam characteristics).

FIG. 7 illustrates an example application where device 100 with ultrasound transducer 110 transmits an acoustic beam into a slice of the brain 4. In conventional ultrasound imaging, the design of medical ultrasound transducers emphasizes the production of a highly focused beam. A highly focused beam, characterized by a small point spread function (PSF), is designed for higher-resolution images or very localized flow data, a typical goal in medical ultrasound. However, as noted previously, detection of CBF using conventional TCD ultrasound is time-consuming, taking up to several minutes, due to the need to make subtle adjustments in the angle and location of the transducer on the skull to allow pinpoint measurements with a higher spatial resolution to ensure the CBF is measured only from a specific artery of interest. Such high spatial resolution is necessary for detailed examinations of the cerebral circulation typically performed using conventional TCD ultrasound transducers.

However, in example time-critical applications, such as CPR, it is unnecessary to know the exact artery from which CBF is detected, only whether adequate diastolic flow is present. As a result, embodiments of the transducers 110 for the device 100 may intentionally utilize as large of a beam as possible (large PSF) in order to capture flow from a broad area while maintaining adequate signal-to-noise characteristics. A larger beam may have a lower spatial resolution that may allow viewing of blood flow over a large volume of the brain. The larger beam may also permit faster acquisition of any flow within the target area, which is critical for emergency care. The larger beam and PSF of the transducer may be tuned using parameters such as, for example, transducer diameter, transducer mechanical focusing, transmit frequency, and pulse length.

Applicant's experience indicates a detection speed is required to be rapid enough for adoption with emergency service providers. In some embodiments, the transducer 110 may be capable of detecting CBF and determining the quality parameters to provide feedback from the captured ultrasound data signals within 3 seconds or less. In other embodiments, the transducer 110 may be capable of detecting CBF and determining the quality parameters from the captured ultrasound data signals within 10 seconds or less. In further embodiments, the transducer 110 may be capable of detecting CBF and determining the quality parameters from the captured ultrasound data signals within 60 seconds or less. This timing may allow the device 100 to be adopted into fast-paced workflows, such as emergency CPR, to enable rapid, compression-by-compression feedback on the quality of CPR (e.g., presence or absence of diastolic flow), where the performance of conventional TCD transducers would not be feasible.

For example, some conventional ultrasound transducers are more tightly focused, with a focal depth of approximately 40 mm to 60 mm, and a minimum −6 decibel (dB) beam width of approximately 2 mm to 4.5 mm. In contrast, to meet the timing requirements above, the example embodiments for the transducer 110 disclosed herein may be designed to utilize a broad-field beam pattern focused on a larger area for a faster acquisition of CBF within a designated target area (i.e., a large PSF). In some embodiments, the example transducer 110 may be unfocused, and may produce a beam having a minimum-6 dB beam width between approximately 3 mm and approximately 6 mm. In other embodiments, the example transducer 110 may be focused, and may produce a beam having a focal depth in a range from approximately 40 mm to approximately 60 mm, and a minimum −6 dB beam width between approximately 2.5 mm and 5 mm. Other desired beam widths may be designed by altering the transducer diameter, focal length, and frequency.

In some embodiments, the transducer 110 may also use a lower frequency than conventional TCD ultrasound to reduce attenuation and more easily detect CBF in individuals with greater physiological variability. The variability may depend on demographic factors including, for example, age, gender, and race/ethnicity. In some embodiments, the example transducer 110 may use a frequency in a range from approximately 1.0 megahertz (MHz) to approximately 2.0 MHz. In other embodiments, the example transducer 110 may use a lower frequency of approximately 1.0 MHz.

Instead of a conventional ultrasound display (which is often cumbersome and external to the device with the ultrasound transducer), the display 124 (refer to FIGS. 2-4) of example device 100 may show targeted, real-time data with visual indicators. The display may be, for example, a Liquid Crystal Display (LCD) integrated into the handle 120 of the device. For example, the display 124 may show changes occurring in brain perfusion in response to alterations in applied CPR parameters that would be suitable feedback to first responders. FIGS. 8 and 9 illustrate potential examples of different display outputs 140, 150 that may be shown on the display 124 of the device 100.

FIG. 8 shows an example display output 140 containing quality parameters. The display 124 may show, for example, a display output 140 presenting the current values of flow velocities in the sampled region of the brain, as derived from the TCD data. The ultrasound signals may be filtered and analyzed by the processor 126. The processor 126 may, for example, calculate flow metrics and diastolic sufficiency, and provide an output for the display in the form of one or more quality parameters indicative of the condition of CBF. The display output 140 shows a readout of anterograde flow velocities 142 and retrograde flow velocities 144. The display output 140 may also contain one or more color bars 146 having a gradient scale corresponding to where the flow velocity values fall versus a range of expected, acceptable, and or historical values. Values indicative of comparably positive flow results (i.e., successful cerebral perfusion) may be shown in green, for example, while values indicative of comparably poor and/or ineffective results may be shown in orange or red. Display output 140 may also have LED indicators, audio alarms, concentration maps, and/or other visual data that can be easily interpreted by an operator and assimilated into practice.

FIG. 9 shows another example display output 150 containing a plot of spectral data 152 that may be shown instead of, or in addition to, the data shown in the example display output 140 of FIG. 8. Spectral TCD data 152 may be, for example, velocity versus time graphs that have been demodulated and filtered from raw velocity spectra by the processor 126. Depending on operator preferences, a spectral plot with applied filters may be the preferred method for the presentation of CBF, especially when shown with other quality parameters like the anterograde and retrograde flow velocities 142, 144 from FIG. 8. A plot of spectral data 152 (which may be accompanied by a scale showing relative intensity) may also make it easier to ascertain whether a change in one or more of excitation power, attenuation, monitoring depth, sample volume, gain, and/or wall filter for the device 100 is necessary based on the condition of the subject.

It may sometimes be useful to process and visualize CBF data on assets remote from the device 100. In some examples, example device 100 may be operably connected to a remote receiver 160 (e.g., in communication with the receiver 160) by the communication module 128. Remote receiver 160 may be any combination of assets such as processors, data centers, controllers, large visual displays, audio filters, media storage, and/or other devices. The communications between the communication module 128 and the remote receiver 160 may be one-way or two-way, for example, using a network to facilitate data transfer. Depending on the type of data to be transmitted, communication between the communication module 128 and the remote receiver 160 may be handled by, for example, electrical conductors and/or optical cables that may be configured to plug into the device 100. In some examples, communication between the communication module 128 and the remote receiver 160 may be wireless via RF (e.g., Bluetooth, mobile broadband, etc.), optical methods, infrared methods, or other suitable communications means. In some examples, the communications module 128 may allow the device to automatically generate and send signals to an emergency department via a secure mobile network to provide information to clinicians in advance of a subject's arrival.

FIG. 10 illustrates an example embodiment in which the remote receiver 160 includes a mechanical housing with a visual display 164 or a software interface to display a visual display 164 on the monitor of a tablet, computer, or other electronic device. The visual display 164 may be configured to show a number of different outputs simultaneously or in sequence. In one example, displays 164 may be a magnified view on an external screen mirroring what is shown on the display 124 of the device 100. The external screen can be one or more of a screen in the mechanical housing of the remote receiver or a display of a mobile device, a tablet, a computer, or other electronic device. In another example, displays 164 may show scaled plots of spectral data 162 that has been processed, amplified, and/or filtered by a processor of the remote receiver 160. In a further example, the visual display 164 may show data structures 166 and/or other data supplemental information. Data structure 166 (e.g., a table, list, function, etc.) may be used to estimate and compare flow metrics, CPR parameters, and other information derived at least in part on CBF signals from the transducer 110. In some embodiments, the CBF signals may be supplemented by physical properties measured with the one or more other sensors in tandem used in, for example, life support protocols. The data structures 166 may also include a lookup tables or other means to demonstrate a subject's condition compared to applicable standards and metrics. These comparisons may be used in conjunction with those provided by the handheld device 100 to inform treatment decisions.

Referring to the example shown in FIG. 10, spectral data 162 displayed as a TCD spectrogram (velocity vs. time) may indicate that, due to ineffective CPR, blood was moving forwards during the systole and backwards during the diastole, indicating a lack of positive diastolic flow in spectrum A (only positive velocities are plotted, note regions showing zero, or negative, flow velocity). Based on this real-time feedback from the device 100, operators may take action and some CPR parameters may be adjusted, such as altering the depth of compressions. After adjustments to CPR parameters, CBF may be observed from the device 100 as a restoration of sufficient blood flow to the brain, as shown in spectrum B of FIG. 10 (note the lack of regions showing zero, or negative, flow velocity). By utilizing such feedback, better subject outcomes may be experienced with fewer neurological deficits and increased subject survival of, for example, cardiac arrest.

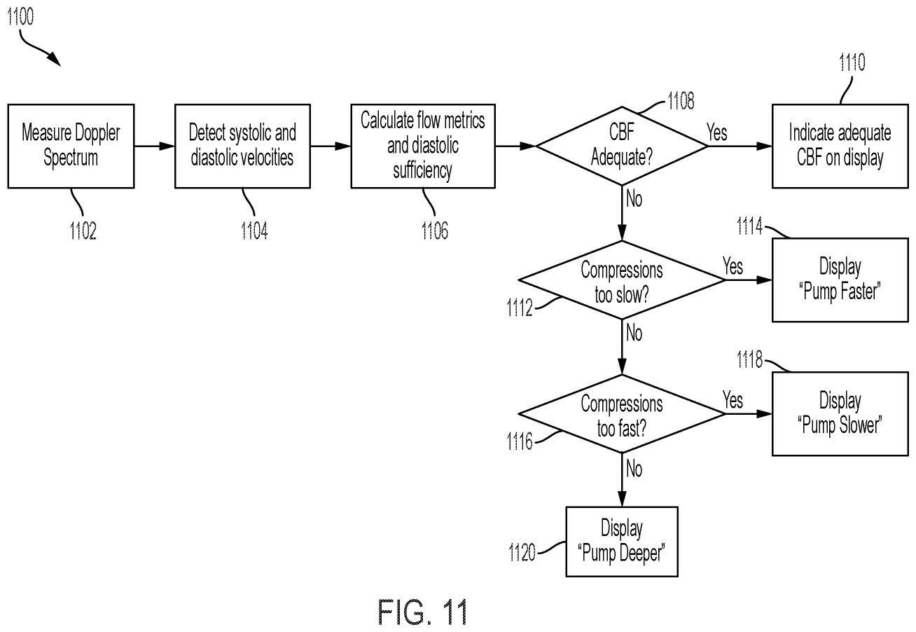

FIG. 11 is a brief visualization of a procedural flow of a process 1100 for providing CBF feedback from any embodiments of the device 100 while performing an emergency procedure, such as CPR. The visualization represents a sequence of operations and/or decisions as an example and is not intended to be limiting. During CPR compressions, an operator may use the device 100 to measure a Doppler ultrasound spectrum from the transducer 110 of the device 100, as indicated by block 1102. From the spectrum data, systolic and diastolic velocities in the brain may be detected or measured, as indicated by block 1104. At block 1106, the systolic and diastolic velocities may be used (for instance, by device processor 126) to calculate flow metrics and diastolic sufficiency, for example, as described herein.

When the flow metrics have been calculated and the condition of CBF ascertained, the device 100 and/or operators may determine, at block 1108, whether CBF is adequate for perfusion for patient survival and to limit the risk of a neurological deficit. The determination may be based on, for example, a comparison of CBF data with known AHA guidelines, a set of known baseline healthy flow metrics, and/or other guidelines preferred by the operator. Responsive to a determination that CBF is adequate based on the guidelines, at block 1110, adequate flow may be indicated on the display 124 of the device 100 for the current execution of CPR or other operation.

Responsive to a determination at block 1108 that CBF is inadequate based on the guidelines, at block 1112, a check may be performed to determine whether the speed of compressions is appropriate for the condition of the subject. For example, responsive to a determination that the compressions are too slow at block 1114, the display 124 of the device 100 may display a message or otherwise indicate the recommendation to speed the pace of compressions, as indicated by block 1114. Responsive to a determination that the compressions are too fast at block 1116, the display 124 of the device 100 may display a message or otherwise indicate the recommendation to slow the pace of compressions, as indicated by block 1118. Responsive to a determination that CBF is inadequate at block 1108 based on the guidelines and that the pace of compressions is appropriate at blocks 1112 and 1116, the display 124 of the device 100 may display a message or otherwise indicate the recommendation to increase the depth of compressions, as indicated by block 1120.

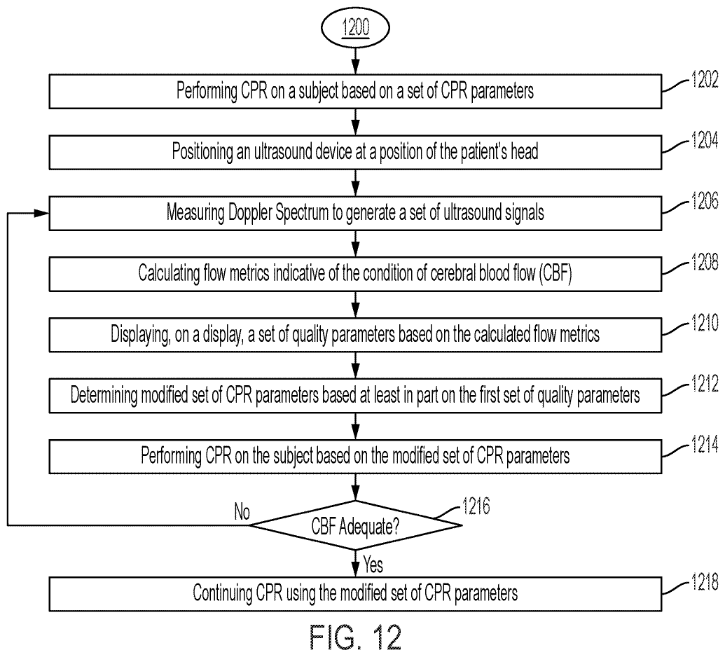

FIG. 12 shows an alternate illustration for a method for performing CPR based on monitored CBF, for example, as described herein. The example method 1200 is illustrated as a collection of blocks in a logical flow graph, which represent a sequence of operations. In some embodiments of the method 1200, one or more of the blocks may be manually and/or automatically executed. The order in which the operations are listed and described is not intended to be construed as a limitation, and any number of the described blocks may be combined in any order and/or in parallel to implement the method.

The example method 1200, at 1202, may include performing CPR on a subject based on a set of CPR parameters, for example, as described herein. In some embodiments, the CPR parameters may be based on AHA guidelines and/or other recommendations, and may include rate of compressions, depth of compressions administered, duty cycle, inter-compression pause time, and release velocity. The CPR may be performed in an emergency hospital, other hospital settings, or out-of-hospital environment, for example, as described herein.

At 1204, the example method 1200 may include positioning an ultrasound device at a position of the patient's head, for example, as described herein. The ultrasound device may be a portable device utilizing a large, broad-field acoustic beam. In some examples, the ultrasound device may be capable of determining CBF in 30 seconds or less, enabling real-time treatment decisions to be based directly on the CBF measurements. In some embodiments, device may have an ultrasound transducer with a sensor head which may be placed flush against a subject's head. The position for the ultrasound device may be based on, for example, operator procedures or the position may be chosen based on a subject's symptomatic response at the time of treatment.

At 1206, the example method 1200 may include measuring a Doppler spectrum to generate a set of ultrasound signals. The signals may be for example, a spectral plot and/or tabulated data as described herein. At 1208, the example method may include calculating flow metrics indicative of the condition of CBF. The calculations may be performed, by a processor of the ultrasound device, for example, as described herein.

The example method 1200 at 1210 may include displaying, on a display of the device, a set of quality parameters based on the calculated flow metrics, for example, as described herein. The display may be an LCD or similar visual display media mounted integral to the handle of the device. The quality parameters may include for example, anterograde blood flow velocities, retrograde blood flow velocities, plots of transcranial Doppler spectral data, and/or other metrics. In some embodiments, the operator of the device may selectively determine which quality parameters the operator would like to be displayed on the display of the device.

In some embodiments, the ultrasound device may also include a communications module for communicating the ultrasound data to a receiver remote from the device. The remote receiver may include, but is not limited to, one or more displays, processors, data storage media, ultrasound stages, and similar devices. In some examples, the remote receiver may have filtering, amplification, and/or display capabilities to supplement those of the ultrasound device. In one example, the remote receiver may receive the communicated ultrasound signals and display a data structure on an external display. The data structure may include tabulated CBF data, spectral plots, and/or lookup tables containing sets of known hemodynamic targets and CPR parameters, for example, as described herein.

At 1212, the example method 1200, may include determining a modified set of CPR parameters based at least in part on the first set of quality parameters, for example, as described herein. For example, the quality parameters may indicate feedback that CBF is insufficient and that adjustments to the CPR parameters are recommended (e.g., change in the rate of compressions, change in the depth of compressions, etc.). At 1214, the example method 1200, may then include performing CPR on the subject based on the modified set of CPR parameters. The example method 1200 at 1216 may then involve determining whether CBF is adequate to promote perfusion based on the adjusted CPR being administered. For example, the determination may be made at least partially by a comparison of CBF measurements from the ultrasound device with known hemodynamic targets, a healthy baseline, or other metrics such as those as described herein.

For example, responsive to determining that CBF is inadequate for successful perfusion, the example method 1200 at 1216 may involve returning to 1206 to generate new ultrasound signals based on the administered modified set of CPR parameters. For example, responsive to determining that CBF is adequate for successful perfusion, the example method 1200 at 1218 may include continuing CPR using the modified set of parameters so that perfusion may continue and so that the chances of patient survival from, for example, cardiac arrest is improved, and the risk of neurological damage is limited.

In some embodiments, the determination of the additional or modified set of CPR parameters may be based, at least in part, on the first set of quality parameters. For example, a sequence of flow metrics and quality parameters may be calculated based on CBF data obtained while administering a current set of CPR parameters and compared with the previous set of flow metrics and quality parameters to determine the fidelity and effectiveness of the CPR. Real-time recommendations on adjustments to the CPR parameters may then be made, for example, as described herein. Similarly, TCD readings on CBF as a result of the adjustments to the CPR parameters may be taken, and the resulting calculated flow metrics and quality parameters compared with the previous values to determine a new set of CPR parameters.

In some embodiments, the determination of the additional or modified set of CPR parameters may involve moving the ultrasound device and collecting CBF data from a second location on the subject's head different from the first location. A second set of flow metrics and quality parameters may then be calculated at the second location and compared with those from the first location. Based at least in part on the comparison of CBF data between the first location and the second location, adjustments to the CPR parameters may be recommended, for example, as described herein.

In some embodiments, the ultrasound device may include an audio device configured to generate an audio output based on a comparison of the one or more quality parameters with a set of known hemodynamic targets. The audio device may be, for example, a speaker that can be in communication with a processor of the ultrasound device, for example, as described herein. When a new set of CPR parameters is determined in the example method 1200, the audio device may alert an operator with simplified audio messages in accordance with second set of parameters. For example, the audio device may audibly alert an operator to “Pump Faster” when compressions are determined to be too slow. Similarly, the audio device may audibly alert an operator to “Pump Slower” when compressions are determined to be too fast. Responsive to determining that CBF remains inadequate and the compression rate is acceptable, the audio device may audibly alert an operator to “Pump Deeper.” Other alerts of importance to a procedure may also be contemplated.

In some embodiments, the ultrasound device of method 1200 may also include at least one energy storage device as an electrical power source. The energy storage device may, for example, used to provide electrical power to one or more of the processor, the display, the ultrasound transducer, the communications module, and the audio device. In some examples, the energy storage device may be a rechargeable battery although other methods known in the art for storage and transmission of electrical power may also be used.

Although the examples illustrated in FIG. 11 and FIG. 12 are provided in the context of an emergency CPR procedure for clarity, it can be appreciated that the use of the example device 100 to monitor CBF may also be applied to other time-critical procedures, for example, as described herein. Those of skill in the art will recognize that for other examples, the method steps and terminology may change accordingly.

Having now described some illustrative embodiments of the disclosure, it should be apparent to those skilled in the art that the foregoing is merely illustrative and not limiting, having been presented by way of example only. Numerous modifications and other embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Those skilled in the art should appreciate that the parameters and configurations described herein are exemplary and that actual parameters and/or configurations will depend on the specific application in which the systems, methods, and/or aspects or techniques of the disclosure are used. Those skilled in the art should also recognize or be able to ascertain, using no more than routine experimentation, equivalents to the specific embodiments of the disclosure. It is, therefore, to be understood that the embodiments described herein are presented by way of example only and that, within the scope of any appended claims and equivalents thereto, the disclosure may be practiced other than as specifically described.

Furthermore, the scope of the present disclosure shall be construed to cover various modifications, combinations, additions, alterations, etc., above and to the above-described embodiments, which shall be considered to be within the scope of this disclosure. Accordingly, various features and characteristics as discussed herein may be selectively interchanged and applied to other illustrated and non-illustrated embodiments, and numerous variations, modifications, and additions further may be made thereto without departing from the spirit and scope of the present disclosure as set forth in the appended claims.

What is claimed is:

1. A device comprising:
a transducer for monitoring cerebral blood flow (CBF) in a subject, the transducer configured to generate ultrasound signals indicative of CBF conditions in the subject;
a handle attached to the transducer, the handle configured for an operator to manipulate the transducer with one hand; and
a display integral with the handle, the display configured to present one or more quality parameters determined from the ultrasound signals;
wherein the one or more quality parameters determined from the ultrasound signals are indicative of presence and strength of diastolic flow in the subject.

2. The device of claim 1, wherein the one or more quality parameters displayed comprise at least one of:
anterograde blood flow velocities;
retrograde blood flow velocities; and
plots of transcranial Doppler spectral data.

3. The device of claim 1, wherein the device further comprises speaker configured to generate an audio output for the operator, the audio output based on a comparison of the one or more quality parameters with a set of known hemodynamic targets.

4. The device of claim 1, wherein the device is configured to calculate and display one or more quality parameters determined from a specified ultrasound signal of the ultrasound signals from the transducer within 30 seconds or less of generation of the specified ultrasound signal.

5. The device of claim 4, wherein a plurality of quality measurements is used by the operator to alter a set of cardiopulmonary resuscitation (CPR) parameters being administered to the subject, the CPR parameters comprising at least one of a compression depth, a compression rate, a duty cycle, an inter-compression pause time, and a release velocity.

6. The device of claim 1, wherein the device comprises a communications module configured to communicate the ultrasound signals to a receiver remote from the device.

7. The device of claim 1, wherein the device is portable and configured to provide the CBF conditions in an out-of-hospital location.

8. The device of claim 1, further comprising a processor configured to process and perform calculations on the ultrasound signals.

9. The device of claim 1, further comprising an energy storage device as a power source.

10. A method for performing cardiopulmonary resuscitation (CPR) based on monitored cerebral blood flow (CBF), the method comprising:
performing CPR on a subject, the CPR performed based on a first set of CPR parameters;
positioning an ultrasound device comprising an ultrasound transducer at a first position on a patient;
measuring a Doppler spectrum to generate a first set of ultrasound signals from the ultrasound transducer;
calculating flow metrics and diastolic sufficiency indicative of a first condition of CBF;
displaying a first set of quality parameters on a display of the ultrasound device based on the first condition of CBF;
determining a second set of CPR parameters based at least in part on the first set of quality parameters; and
performing CPR on the subject based on the second set of CPR parameters.

11. The method of claim 10, further comprising:
generating a second set of ultrasound signals responsive to the CPR performed at the second set of CPR parameters;
calculating the flow metrics and diastolic sufficiency indicative of a second condition of CBF;
displaying a second set of quality parameters on the display based on the second condition of CBF;
determining a third set of CPR parameters based at least in part on a change between the first set of quality parameters and the second set of quality parameters; and
performing CPR on the subject based on the third set of CPR parameters.

12. The method of claim 11, further comprising continuing CPR at the third set of CPR parameters when the second condition of CBF is indicative of adequate blood flow in a patient's brain.

13. The method of claim 10, further comprising:
positioning the ultrasound device at a second position on the patient different from the first position;
generating a second set of ultrasound signals indicative of a second condition of CBF at the second position responsive to the CPR performed;
calculating and displaying another set of quality parameters on the display based on the second condition of CBF; and
determining, based at least in part on a comparison of the first condition of CBF at the first position and the second condition of CBF at the second position, a difference in diastolic flow between first position and the second position.

14. The method of claim 10, further comprising:
communicating the ultrasound signals to a receiver remote from the ultrasound device.

15. The method of claim 14, further comprising:
receiving the communicated ultrasound signals and displaying, on an external display in communication with the receiver, a data structure comprising at least one of:
information relating to the first condition of CBF from the communicated ultrasound signals; and
a lookup table comprising a set of known hemodynamic targets and CPR parameters.

16. The method of claim 10, wherein the displaying of one or more quality parameters comprises displaying at least one of:
anterograde blood flow velocities;
retrograde blood flow velocities; and
plots of transcranial Doppler spectral data.

17. The method of claim 10, wherein the ultrasound device further comprises:
an audio device configured to generate an audio output based on a comparison of the first set of quality parameters with a set of known hemodynamic targets.

18. The method of claim 10, wherein the ultrasound device is portable and configured to provide the first condition of CBF in out-of-hospital locations.

19. The method of claim 10, wherein the display is integral with a handle of the ultrasound device.

20. The method of claim 10, wherein the ultrasound device further comprises:
a processor to perform the calculating of the flow metrics and the diastolic sufficiency.

21. (canceled)
